(19) United States
(12) Patent Application Publication
(10) Pub. No.: US 2025/0161106 A1
(43) Pub. Date: May 22, 2025

(54) Systems and Methods for Agitating a Targeted Temperature Management Fluid

(71) Applicant: C. R. Bard, Inc., Franklin Lakes, NJ (US)

(72) Inventors: Gabriel A. Johnston, Broomfield, CO (US); Madeline Stich, Thornton, CO (US); Rebecca D. Salisbury, Lafayette, CO (US); Sean E. Walker, Platteville, CO (US)

(21) Appl. No.: 18/840,855

(22) PCT Filed: Feb. 25, 2022
(86) PCT No.: PCT/US2022/018008

§ 371 Date: Aug. 22, 2024

(51) Int. Cl. A61F 7/00; A61F 7/02

(52) U.S. Cl. CPC A61F 7/0085; A61F 7/02; A61F 2007/0054

(57) ABSTRACT

Disclosed herein are systems and methods for providing targeted temperature management (TTM) therapy to a patient. Systems included herein provide an airflow to the patient in additional to a fluid flow that define a thermal energy exchange with the patient. Various systems may provide air at a defined TTM temperature to a thermal contact pad, a mattress or a ventilator that delivers the TTM air to the patient via the ventilation therapy. Also disclosed herein are systems, devices, and methods for preventing, managing, and/or removing perspiration moisture from between the thermal contact pad and the patient. Disclosed herein is a thermal contact pad includes a wicking material to draw moisture away from the patient. Disclosed herein also is a thermal contact pad including airflow that draws moisture away from the patient.

### BACKGROUND

The effect of temperature on the human body has been well documented and the use of targeted temperature management (TTM) systems for selectively cooling and/or heating bodily tissue is known. Elevated temperatures, or hyperthermia, may be harmful to the brain under normal conditions, and even more importantly, during periods of physical stress, such as illness or surgery. Conversely, lower body temperatures, or mild hypothermia, may offer some degree of neuroprotection. Moderate to severe hypothermia tends to be more detrimental to the body, particularly the cardiovascular system.

Targeted temperature management can be viewed in two different aspects. The first aspect of temperature management includes treating abnormal body temperatures, i.e., cooling the body under conditions of hyperthermia or warming the body under conditions of hypothermia. The second aspect of thermoregulation is an evolving treatment that employs techniques that physically control a patient's temperature to provide a physiological benefit, such as cooling a stroke patient to gain some degree of neuroprotection. By way of example, TTM systems may be utilized in early stroke therapy to reduce neurological damage incurred by stroke and head trauma patients. Additional applications include selective patient heating/cooling during surgical procedures such as cardiopulmonary bypass operations.

TTM systems circulate a fluid (e.g., water) through one or more thermal contact pads coupled to a patient to affect surface-to-surface thermal energy exchange with the patient. In general, TTM systems include a TTM fluid control module coupled to at least one contact pad via a fluid deliver line. One such TTM system is disclosed in U.S. Pat. No. 6,645,232, titled “Patient Temperature Control System with Fluid Pressure Maintenance” filed Oct. 11, 2001, and one such thermal contact pad and related system is disclosed in U.S. Pat. No. 6,197,045 titled “Cooling/heating Pad and System” filed Jan. 4, 1999, both of which are incorporated herein by reference in their entireties. As noted in the '045 patent, the ability to establish and maintain intimate pad-to-patient contact is of importance to fully realizing medical efficacies with TTM systems.

In some instances of a TTM therapy, the TTM fluid may be portions of a thermal contact pad where the TTM fluid flow is low or stagnant resulting in a decreased thermal energy with the patient. Disclosed herein are TTM systems, thermal contact pads, and devices that promote increased thermal exchange efficiency with the patient.

### SUMMARY

Briefly summarized, disclosed herein is a targeted temperature management (TTM) system for exchanging thermal energy with a patient, according to some embodiments. The TTM system includes a TTM module configured to provide a TTM fluid at a defined fluid temperature in accordance with a TTM therapy and one or more thermal-contact pads fluidly coupled with the TTM module via a fluid delivery line (FDL) extending between the TTM module and the pad, where the pad is configured to receive the TTM fluid from the TTM module, and circulate the TTM fluid within fluid channels of the pad to define a thermal energy exchange between the TTM fluid and the patient. The system further includes a fluid agitator operatively coupled with the TTM fluid, the fluid agitator configured to cause an agitation of the TTM fluid within the pad.

In some embodiments, the agitator causes an oscillation of the TTM fluid at a frequency greater than 20 KHz. According to some embodiments, the agitator is coupled with the TTM fluid at the pad or within a TTM fluid supply tank of the TTM module.

In some embodiments, the pad includes a fluid flow disrupting mechanism configured to inhibit low-flow conditions of the TTM fluid within the flow channels of the pad. The disrupting mechanism may include a number of fluid flow disrupting members protruding within the flow channels, where the disrupting members are configured to enhance a TTM fluid flow velocity through otherwise low-flow areas of the flow channels.

In some embodiments, at least a first subset of the number of disrupting members are configured to deflect in response to a force applied by the TTM fluid flow. In some embodiments, at least a second subset of the number of disrupting members are configured to rotate in response to a torque applied by the TTM fluid flow.

In some embodiments, at least a third subset of the number of disrupting members are formed of a material having a thermal conductivity greater than a thermal conductivity of the TTM fluid and in further embodiments, at least a fourth subset of the number of disrupting members are formed of a material having a thermal compacity greater than a thermal compacity of the TTM fluid.

In some embodiments, the flow channels include a spiral flow path extending between a first end located at a central portion of the spiral and a second end located at a perimeter of the spiral.

In some embodiments, the fluid flow disrupting mechanism includes a number of orifices extending through an exterior wall of the pad between the TTM fluid and the environment, where a negative pressure of the TTM fluid within the flow channels draws air through the orifices into the flow channels causing air bubbles within the flow channels, and where the air bubbles cause a flow disturbance of the TTM fluid to agitate the TTM fluid.

In some embodiments, the TTM fluid includes a surfactant to enhance a thermal energy exchange between the TTM fluid and an inside surface of the flow channels.

Also disclosed herein is a targeted temperature management (TTM) system for exchanging thermal energy with a patient according to further embodiments. The TTM system includes a TTM module configured to provide a TTM fluid at a defined fluid temperature in accordance with a TTM therapy and one or more thermal-contact pads fluidly coupled with the TTM module via a fluid delivery line (FDL) extending between the TTM module and the pad, where the pad is configured to receive the TTM fluid from the TTM module, and circulate the TTM fluid within flow channels of the pad to define a thermal energy exchange between the TTM fluid and the patient. The pad includes a fluid flow disrupting mechanism configured to inhibit low-flow conditions of the TTM fluid within the flow channels of the pad.

In some embodiments, the disrupting mechanism includes a number of fluid flow disrupting members protruding within the flow channels, where the disrupting members are configured to enhance a TTM fluid flow velocity through otherwise low-flow areas of the flow channels.

In some embodiments, at least a first subset of the number of disrupting members are configured to deflect in response to a force applied by the TTM fluid flow. In some embodiments, at least a second subset of the number of disrupting members are configured to rotate in response to a torque applied by the TTM fluid flow.

In some embodiments, at least a third subset of the number of disrupting members are formed of a material having a thermal conductivity greater than a thermal conductivity of the TTM fluid and in further embodiments, at least a fourth subset of the number of disrupting members are formed of a material having a thermal compacity greater than a thermal compacity of the TTM fluid.

In some embodiments, the flow channels include a spiral flow path extending between a first end located at a central portion of the spiral and a second end located at a perimeter of the spiral.

In some embodiments, the fluid flow disrupting mechanism includes a number of orifices extending through an exterior wall of the pad between the TTM fluid and the environment, where a negative pressure of the TTM fluid within the flow channels draws air through the orifices into the flow channels causing air bubbles within the flow channels, and where the air bubbles cause a flow disturbance of the TTM fluid to agitate the TTM fluid.

In some embodiments, the TTM fluid includes a surfactant to enhance a thermal energy exchange between the TTM fluid and an inside surface of the flow channels.

In some embodiments, the system further includes a fluid agitator operatively coupled with the TTM fluid, the fluid agitator configured to cause an agitation of the TTM fluid within the pad. In some embodiments, the agitator causes an oscillation of the TTM fluid at a frequency exceeding 20 KHz, and the agitator may be coupled with the TTM fluid at the pad.

Also disclosed herein is a method of exchanging thermal energy with a patient, according to some embodiments. The method includes (i) circulating, by a targeted temperature management (TTM) system, a TTM fluid within a thermal contact pad applied to the patient, the TTM fluid having a temperature defined by the TTM module in accordance with a TTM therapy and (ii) agitating the TTM fluid within flow channels of the pad to enhance a thermal energy exchange between the TTM fluid and the patient.

In some embodiments of the method, the TTM system includes a fluid agitator operatively coupled with the TTM fluid, where the fluid agitator is configured to cause the agitation of the TTM fluid within the pad.

In some embodiments of the method, the pad includes a fluid flow disrupting mechanism configured to inhibit low-flow conditions of the TTM fluid within flow channels of the pad.

In some embodiments of the method, the fluid flow disrupting mechanism includes a number of fluid flow disrupting members protruding within the flow channels, where the disrupting members are configured to enhance a TTM fluid flow velocity through otherwise low-flow areas of the flow channels.

In some embodiments, the method further includes deflecting at least a first subset of the number of disrupting members, the deflection resulting from a force applied by the TTM fluid flow, and in some embodiments, the method includes rotating at least a second subset of the number of disrupting members, where the rotation results from a torque applied by the TTM fluid flow.

In some embodiments of the method, at least a third subset of the number of disrupting members are formed of a material having a thermal conductivity greater than a thermal conductivity of the TTM fluid and in some embodiments of the method, at least a fourth subset of the number of disrupting members are formed of a material having a thermal compacity greater than a thermal compacity of the TTM fluid.

In some embodiments of the method, the disrupting mechanism includes a number of orifices extending through an exterior wall of the pad between the TTM fluid and the environment, and the method further includes drawing air through the orifices into the flow channels via a negative pressure of the TTM fluid within the flow channels, where the air bubbles cause a flow disturbance of the TTM fluid to agitate the TTM fluid.

In some embodiments of the method, the TTM fluid includes a surfactant to enhance a thermal energy exchange between the TTM fluid and an inside surface of the flow channels.

These and other features of the concepts provided herein will become more apparent to those of skill in the art in view of the accompanying drawings and the following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a patient being treated by targeted temperature management (TTM) system to cool or warm the patient, in accordance with some embodiments;

FIG. 2 illustrates a hydraulic schematic of the TTM system of FIG. 1, in accordance with some embodiments;

FIG. 3 illustrates a block diagram depicting various elements of a console of the TTM module of FIG. 1, in accordance with some embodiments;

FIG. 4A is a top view illustration of the thermal contact pad of FIG. 1 showing the flow channels for the TTM fluid, in accordance with some embodiments;

FIG. 4B illustrates a detailed top view of a portion of the thermal contact pad of FIG. 4A showing an optional flow diverter protruding within the flow channels, in accordance with some embodiments;

FIG. 4C illustrates a detailed top view of a portion of the thermal contact pad of FIG. 4A showing an optional deflectable flow diverter protruding within the flow channels, in accordance with some embodiments;

FIG. 4D illustrates a detailed top view of a portion of the thermal contact pad of FIG. 4A showing an optional rotatable flow diverter protruding within the flow channels, in accordance with some embodiments;

FIG. 4E illustrates a cross-sectional side view of a portion of the thermal contact pad of FIG. 4A cut along section lines 4E-4E showing an optional number of orifices allowing air to enter the flow channels, in accordance with some embodiments;

FIG. 5 illustrates a detailed top view of a portion of the thermal contact pad of FIG. 4A showing an optional radiused corners number of the flow channels, in accordance with some embodiments; and

FIG. 6 illustrates an optional spiral arrangement of the flow channels that may be incorporated into the thermal contact pad of FIG. 1, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. The words “including,” “has,” and “having,” as used herein, including the claims, shall have the same meaning as the word “comprising.” Furthermore, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

The phrases “connected to” and “coupled to” refer to any form of interaction between two or more entities, including mechanical, electrical, magnetic, electromagnetic, fluid, signal, communicative (including wireless), and thermal interaction. Two components may be connected or coupled to each other even though they are not in direct contact with each other. For example, two components may be coupled to each other through an intermediate component.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Unless defined otherwise, all technical and scientific terms used herein have the same meaning as commonly understood by those of ordinary skill in the art.

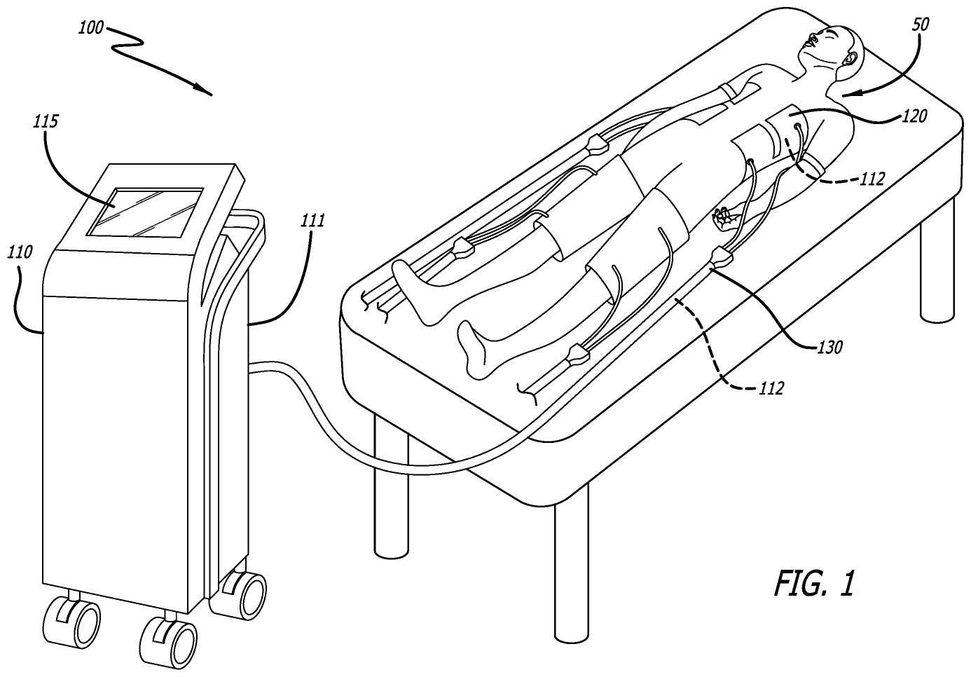

FIG. 1 illustrates a targeted temperature management (TTM) system 100 connected to a patient 50 for administering targeted temperature management therapy to the patient 50 which may include a cooling and/or warming of the patient 50, in accordance with some embodiments. The TTM system 100 includes a TTM module 110 including a graphical user interface (GUI) 115 enclosed within a module housing 111. The TTM system 100 includes a fluid deliver line (FDL) 130 extending from the TTM module 110 to a thermal contact pad (pad) 120 to provide for flow of TTM fluid 112 between the TTM module 110 and the pad 120.

The TTM system 100 may include 1, 2, 3, 4 or more pads 120 and the TTM system 100 may include 1, 2, 3, 4 or more fluid delivery lines 130. In use, the TTM module 110 prepares the TTM fluid 112 for delivery to the pad 120 by heating or cooling the TTM fluid 112 to a defined temperature in accordance with a prescribed TTM therapy. The TTM module 110 circulates the TTM fluid 112 within the pad 120 to facilitate thermal energy exchange with the patient 50. During the TTM therapy, the TTM module 110 may continually control the temperature of the TTM fluid 112 toward a target TTM temperature. In some instances, the target TTM temperature may change during the TTM therapy.

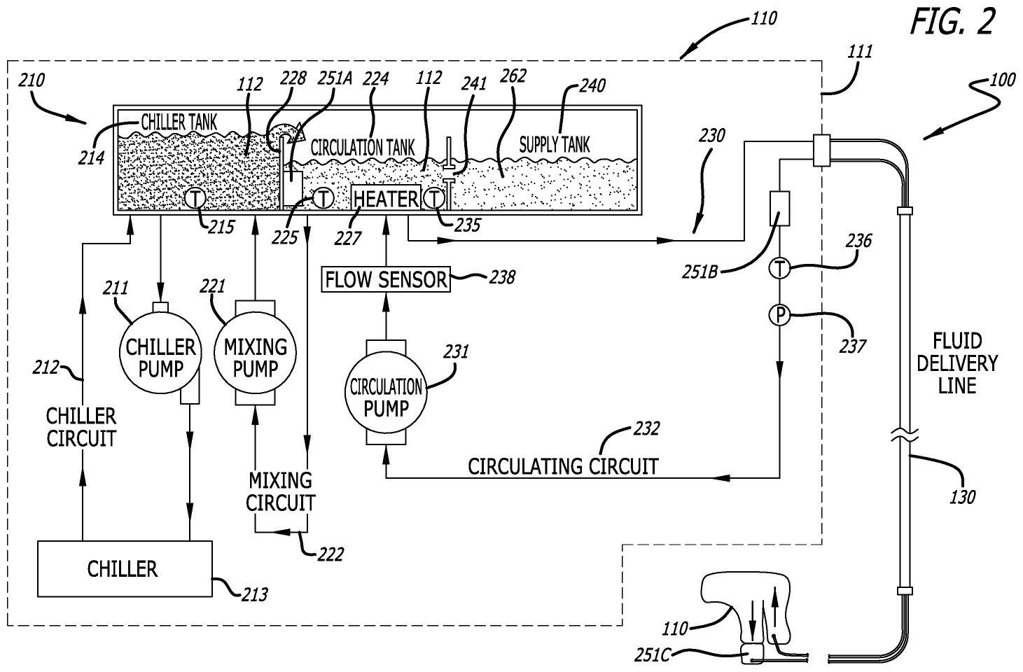

FIG. 2 illustrates a hydraulic schematic of the TTM system 100. The FDL 130 and the pad 120 are disposed external to the housing 111 of the TTM module 110. The TTM module includes various fluid sensors and fluid control devices to prepare and circulate the TTM fluid 112. The fluid subsystems of the TTM module may include a temperature control subsystem 210 and a circulation subsystem 230.

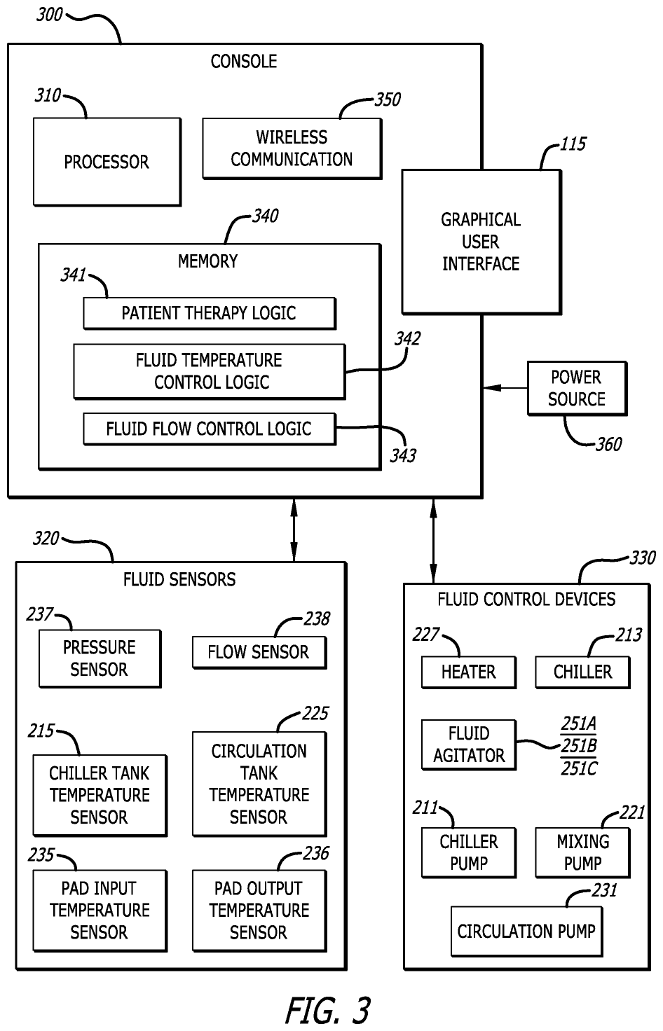

The temperature control subsystem 210 may include a chiller pump 211 to pump (recirculate) TTM fluid 112 through a chiller circuit chiller 212 that includes a chiller 213 and a chiller tank 214. A temperature sensor 215 within the chiller tank 214 is configured to measure a temperature of the TTM fluid 112 within the chiller tank 214. The chiller 213 may be controlled by a temperature control logic (see FIG. 3) as further described below to establish a desired temperature of the TTM fluid 112 within chiller tank 214. In some instances, the temperature of the TTM fluid 112 within the chiller tank 214 may be less than the target temperature for the TTM therapy.

The temperature control subsystem 210 may include may further include a mixing pump 221 to pump TTM fluid 112 through a mixing circuit 222 that includes the chiller tank 214, a circulation tank 224, and a dam 228 disposed between the chiller tank 214 and circulation tank 224. The TTM fluid 112, when pumped by the mixing pump 221, enters the chiller tank 214 and mixes with the TTM fluid 112 within the chiller tank 214. The mixed TTM fluid 112 within the chiller tank 214 flows over the dam 228 and into the circulation tank 224. In other words, the mixing circuit 222 mixes the TTM fluid 112 within chiller tank 214 with the TTM fluid 112 within circulation tank 224 to cool the TTM fluid 112 within the circulation tank 224. A temperature sensor 225 within the circulation tank 224 measures the temperature of the TTM fluid 112 within the circulation tank 224. The temperature control logic may control the mixing pump 221 in accordance with temperature data from the temperature sensor 225 within the circulation tank 224.

The circulation tank 224 includes a heater 227 to increase to the temperature of the TTM fluid 112 within the circulation tank 224, and the heater 227 may be controlled by the temperature control logic. In summary, the temperature control logic when executed by the processor (see FIG. 3) may receive temperature data from the temperature sensor 215 within the chiller tank and the temperature sensor 225 within the circulation tank 224 and control the operation of the chiller 213, the chiller pump 211, the heater 227, and mixing pump 222 to establish and maintain the temperature of the TTM fluid 112 within the circulation tank 224 at the target temperature for the TTM therapy.

The circulation subsystem 230 includes a circulation pump 213 to pull TTM fluid 112 from the circulation tank 224 and through a circulating circuit 232 that includes the fluid delivery line 120 and the pad 120 located upstream of the circulation pump 213. The circulating circuit 232 also includes a pressure sensor 237 to represent a pressure of the TTM fluid 112 within the pad 120. The circulating circuit 232 also includes a temperature sensor 235 within the circulation tank 224 to represent the temperature of the TTM fluid 112 entering the pad 120 and a temperature sensor 236 to represent the temperature of the TTM fluid exiting the pad 120. A flow meter 238 is disposed downstream of the circulation pump 213 to measure the flow rate of TTM fluid 112 through the circulating circuit 232 before the TTM fluid 112 re-enters that the circulation tank 224.

In use, the circulation tank 224, which may be vented to atmosphere, is located below (i.e., at a lower elevation) the pad 120 so that a pressure within the pad 120 is less than atmospheric pressure (i.e., negative) when fluid flow through the circulating circuit 232 is stopped. The pad 120 is also placed upstream of the circulation pump 231 to further establish a negative pressure within the pad 120 when the circulation pump 213 is operating. The fluid flow control logic (see FIG. 3) may control the operation of the circulation pump 213 to establish and maintain a desired negative pressure within the pad 120. A supply tank 240 provides TTM fluid 112 to the circulation tank 224 via a port 241 to maintain a defined volume of TTM fluid 112 within the circulation tank 224.

In some embodiments, although not required, the TTM fluid 112 may include a surfactant 262 to enhance a thermal energy exchange between the TTM fluid 112 and an inside surface of the flow channels within the pad 120. The surfactant 262 may reduce a boundary layer of the TTM fluid 112 within the channels thereby enhancing a thermal energy exchange via convection.

In some embodiments, although not required, the system 100 may be configured to actively agitate (i.e., disturb, cause a turbulence of, or oscillate) the flow of the TTM fluid 112 within the pad 120. Agitation of the TTM fluid 112 within the pad 120 may enhance the thermal energy exchange between the TTM fluid 112 and an inside surface of the flow channels within the pad 120 by reducing stagnation or low-flow conditions of the TTM fluid 112 at various locations within the pad 120. In some embodiments, the system 100 may include one or more agitators operatively coupled with the TTM fluid 112.

According to some embodiments, the system 100 may include a tank agitator 251A located within or operatively coupled with the circulation tank 224 so as to agitate the TTM fluid 112 within the circulation tank 224. The agitation of the TTM fluid 112 within the circulation tank 224 may propagate along the flow path of the TTM fluid 112 including the FDL 130 to the pad 120 resulting in agitation of the TTM fluid 112 within the pad 120.

As an alternative, or in addition, to the tank agitator 251A, the system 100 may include a circulating circuit agitator 251B disposed in line with the circulating circuit 232 with the TTM module 110. Similar to the tank agitator 251A, agitation of the TTM fluid 112 along the circulating circuit 232 may propagate along the flow path of the TTM fluid 112 including the FDL 130 to the pad 120. Due to the hydraulic placement of the circulating circuit agitator 251B between the circulation pump 231 and pad 120, agitation of the TTM fluid 112 by the circulating circuit agitator 251B may more effectively propagate to the pad 120 than the tank agitator 251A.

As an alternative, or in addition, to the tank agitator 251A and or the circulating circuit agitator 251B, the system 100 may include a pad agitator 251C, located at, adjacent to, or within the pad 120. Due to the hydraulic placement of the pad agitator 251C at the pad 120, agitation of the TTM fluid 112 by the pad agitator 251C may more effectively propagate to the pad 120 than the tank agitator 251A or the circulating circuit agitator 251B.

In some embodiments, any of the agitators 251A-251C may cause an oscillation of the TTM fluid at a frequency greater than 20 KHz.

FIG. 3 illustrates a block diagram depicting various elements of the TTM module 110 of FIG. 1, in accordance with some embodiments. The TTM module includes a console 300 including a processor 310 and memory 340 including non-transitory, computer-readable medium. Logic modules stored in the memory 340 include patient therapy logic 341, fluid temperature control logic 342, and fluid flow control logic 343. The logic modules when executed by the processor 310 define the operations and functionality of the TTM Module 110.

Illustrated in the block diagram of FIG. 3 are fluid sensors 320 as described above in relation to FIG. 2. Each of the fluid sensors 320 are coupled to the console 300 so that data from the fluid sensors 320 may be utilized in the performance of TTM module operations. Fluid control devices 330 are also illustrated in FIG. 3 as coupled to the console 300. As such, logic modules may control the operation of the fluid control devices 330 as further described below.

The patient therapy logic 341 may receive input from the clinician via the GUI 115 to establish operating parameters in accordance with a prescribed TTM therapy. Operating parameters may include a target temperature for the TTM fluid 112 which may comprise a time-based target temperature profile. In some embodiments, the fluid temperature control logic 342 may define other fluid temperatures of the TTM fluid 112 within the TTM module 110, such a target temperature for the TTM fluid 112 within the chiller tank 214, for example.

The fluid temperature control logic 342 may perform operations to establish and maintain a temperature of the TTM fluid 112 delivered to the pad 120 in accordance with a predefined target temperature profile. One temperature control operation may include chilling the TTM fluid 112 within the chiller tank 214. The fluid temperature control logic 342 may utilize temperature data from the chiller tank temperature sensor 215 to control the operation of the chiller 213 to establish and maintain a temperature of the TTM fluid 112 within the chiller tank 214.

Another temperature control operation may include cooling the TTM fluid 112 within the circulation tank 224. The fluid temperature control logic 342 may utilize temperature data from the circulation tank temperature sensor 225 to control the operation of the mixing pump 221 to decrease the temperature of the TTM fluid 112 within the circulation tank 224.

Still another temperature control operation may include warming the TTM fluid 112 within the circulation tank 224. The fluid temperature control logic 342 may utilize temperature data from the circulation tank temperature sensor 225 to control the operation of the heater 227 to increase the temperature of the TTM fluid 112 within the circulation tank 224.

The fluid flow control logic 343 may control the operation of the circulation pump 231. As a thermal energy exchange rate is at least partially defined by the flow rate of the TTM fluid 112 through the pad 120, the fluid flow control logic 343 may, in some embodiments, control the operation of the circulation pump 231 in accordance with a defined thermal energy exchange rate for the TTM therapy.

The fluid flow control logic 343 may control the operation of any or all of fluid agitators 251A-252C. As a thermal energy exchange rate may be enhanced by an agitation (i.e., a disturbance, a turbulence, or an oscillation) of the TTM fluid 112 within the pad 120, the fluid flow control logic 343 may, in some embodiments, control the operation of the fluid agitators 251A-252C. For example, the fluid flow control logic 343 may define an oscillation frequency and/or an oscillation magnitude of the fluid agitators 251A-252C. In some embodiments, the oscillation frequency may be in the ultrasound frequency range, i.e., above 20 KHz.

The console 300 may comprise wireless communication capability 350 to facilitate wireless communication with external devices. A power source 360 provides electrical power to the console 300.

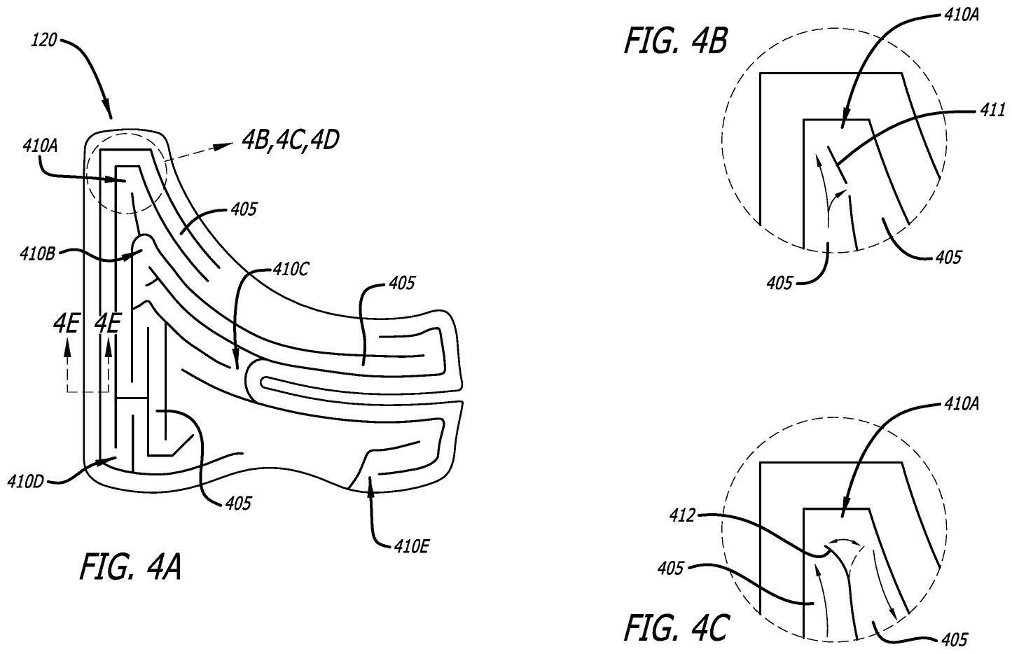

FIG. 4A illustrates a top view of the thermal contact pad 120 of FIG. 1. As shown, the pad 120 includes a series of flow channels 405 to direct the flow of the TTM fluid across the pad 120. As shown, in some embodiments, the flow channels 405 may be arranged to cause a number of low-flow areas, such as the exemplary low-flow areas 410A-410E. As shown, some of the low-flow areas may be located at inside corners of the flow channels 405. The pad 120 generally relies of convective heat transfer to define the thermal energy exchange between the TTM fluid 112 and the inside wall surfaces of the flow channels 405. A low-flow condition may cause a decrease in the convective heat transfer. As such, it may be advantageous to minimize low-flow areas of the flow channels 405.

FIGS. 4B-4E illustrate an optional flow disrupting mechanism that may include various optional elements of the flow channels 405 of the pad 120. The elements may include flow disrupting members disposed or protruding within flow channels 405 of the pad 120. The elements are configured to reduce or inhibit low-flow conditions (including low velocity) of the TTM fluid 112 within the flow channels 405 of the pad 120 and thereby enhance the convective heat transfer. In some embodiments, the elements are configured to cause a turbulence or agitation of the TTM fluid 112 within an otherwise low-flow areas of the flow channels 405.

FIG. 4B illustrates a detailed top view of a portion of the thermal contact pad 120 showing an exemplary flow disrupting member in the form of a flow diverter 411 protruding within the low-flow area 410A of the flow channel 405, in accordance with some embodiments. The flow diverter 411 may be one of a plurality of the flow diverters 411 disposed within low-flow areas. The flow diverter 411 is coupled with one or more channel walls. The flow diverter 411 may be rigidly coupled with the channel 405 so that the flow diverter 411 remains stationary within the flow of the TTM fluid 112. The flow diverters 411 may be configured (i.e., sized, shaped, positioned, and/or oriented) to minimize the low-flow conditions of respective low-flow areas, such as the low-flow area 410A, for example.

In some embodiments, all or a subset of the flow diverters 411 may be formed of a material having a thermal conductivity greater than a thermal conductivity of a channel wall material and/or the TTM fluid 112 so as to enhance the thermal energy exchange between the TTM fluid 112 and the channel wall. In other words, the flow diverter 411 may facilitate thermal energy exchange between the TTM fluid 112 and the channel wall via conductance of heat along the flow diverter 411.

In some embodiments, all or a subset of the flow diverters 411 may be formed of a material having a thermal capacity (i.e., a specific heat) greater than a thermal compacity of a channel wall material and/or the TTM fluid 112 so as to enhance a stability or consistency of the thermal energy exchange between the TTM fluid 112 and the patient 50. In other words, the flow diverter 411 may maintain a substantially constant temperature during the TTM therapy, thereby maintaining a substantially constant temperature of the TTM fluid 112 adjacent the flow diverter 411.

FIG. 4C illustrates a detailed top view of a portion of the thermal contact pad 120 showing an exemplary flow disrupting member in the form of a deflectable flow diverter 412 (e.g., a deflectable flap) protruding within the low-flow area 410A of the flow channel 405, in accordance with some embodiments. The deflectable flow diverter 412 may be one of a plurality of deflectable flow diverters 412 disposed within low-flow areas of the flow channels 405. The deflectable flow diverter 412 is flexibly coupled with one or more channels walls so that the deflectable flow diverter 412 may move/deflect in response to a force exerted thereon by the flow of the TTM fluid 112. In some embodiments, the movement of the deflectable flow diverter 412 may be oscillatory in accordance with an agitation of the flow of the TTM fluid 112. Similar to the flow diverters 411, all or a subset of the deflectable flow diverters 412 may be formed of a material having a thermal conductivity and/or a thermal compacity greater than the channel wall material and/or the TTM fluid 112.

FIG. 4D illustrates a detailed top view of a portion of the thermal contact pad 120 showing an exemplary flow disrupting member in the form of a rotatable flow diverter 413 disposed within the low-flow area 410A of the flow channel 405, in accordance with some embodiments. In some embodiments, the rotatable flow diverter 413 may take the form of a paddle wheel having a plurality of blades extending radially away from a rotational axis of the rotatable flow diverter 413. The rotatable flow diverter 413 may be one of a plurality of rotatable flow diverters 413 disposed within low-flow areas of the flow channels 405. The rotatable flow diverter 413 is rotatably coupled with one or more channels walls so that the rotatable flow diverter 413 rotates in response to a torque applied thereto by the flow of the TTM fluid 112. In some embodiments, the rotation of the rotatable flow diverter 413 may include an oscillation component in accordance with an agitation of the TTM fluid 112. Similar to the flow diverters 411, all or a subset of the rotatable flow diverters 413 may be formed of a material having a thermal conductivity and/or a thermal compacity greater than the channel wall material and/or the TTM fluid 112.

The pad 120 may include any number of flow diverters 411, deflectable flow diverters 412, or rotatable flow diverters 413 alone or in combination. In other words, the pad 120 may include: (i) a first subset of flow disrupting members that includes one or more flow diverters 411; (ii) a second subset the flow disrupting members that includes one or more deflectable flow diverters 412, and/or a third subset the flow disrupting members that includes one or more rotatable flow diverters 413.

FIG. 4E illustrates a cross-sectional side view of a portion of the thermal contact pad 120 showing the optional flow disrupting mechanism that includes a number of orifices 414 that allow air to enter the flow channels 405 forming air bubbles 415 with the TTM fluid 112, in accordance with some embodiments. The presence of the air bubbles 415 may cause a disturbance in the flow of the TTM fluid 112, thereby reducing one or more low-flow conditions along the flow channels 405. A negative pressure of the TTM fluid 112 may draw the air into the flow channels 405 through the orifices 414. The orifices 414 may be disposed along any or all of a top wall 406, a bottom wall 407, or a side wall 408 of the flow channels 405. In some embodiments, a channel wall material and/or size of the orifices 414 may be configured to prevent leakage of the TTM fluid 112 out of the fluid channels 405 due to surface tension of the TTM fluid 112 adjacent the orifices 414. In some embodiments, the orifices 414 may include a check valve (not shown) configured to allow air to flow into the flow channels 405 through orifices 414 and prevent the flow of TTM fluid 112 out of the flow channels 405 through orifices 414.

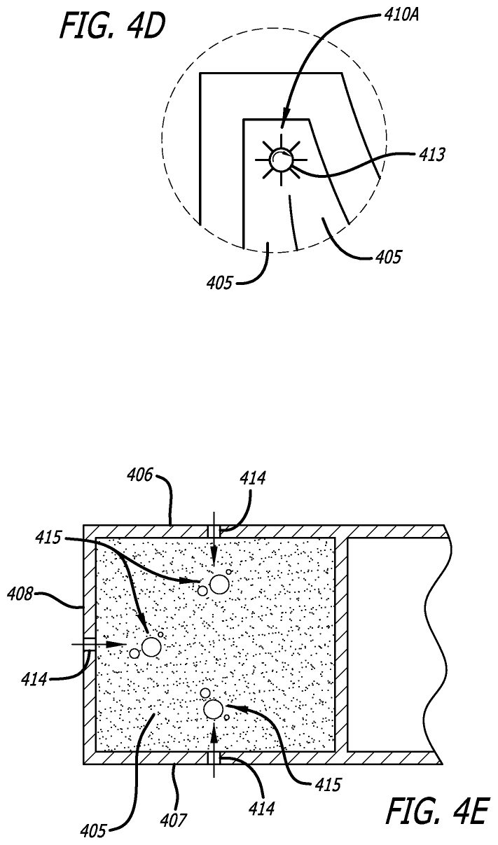

FIG. 5 illustrates a top side view of a portion of the thermal contact pad 120 showing optional radiused corners of number of the flow channels 405, in accordance with some embodiments. In some instances, sharp inside corners of the flow channels 405 may promote a low-flow condition. As such, the pad 120 may optionally include one or more radiused inside corners 505 of the flow channels 405. The radiused inside corners 505 may eliminate pockets of low-flow or stagnant TTM fluid 112. The radiused inside corners 505 may also promote a smooth or constant flow of TTM fluid 112 along the inside corners 505.

Similarly, the pad 120 may optionally include one or more radiused outside corners 506 of the flow channels 405. The radiused outside corners 506 may also promote a smooth or constant flow of TTM fluid 112 around the outside corners further inhibiting low-flow conditions of the TTM fluid 112.

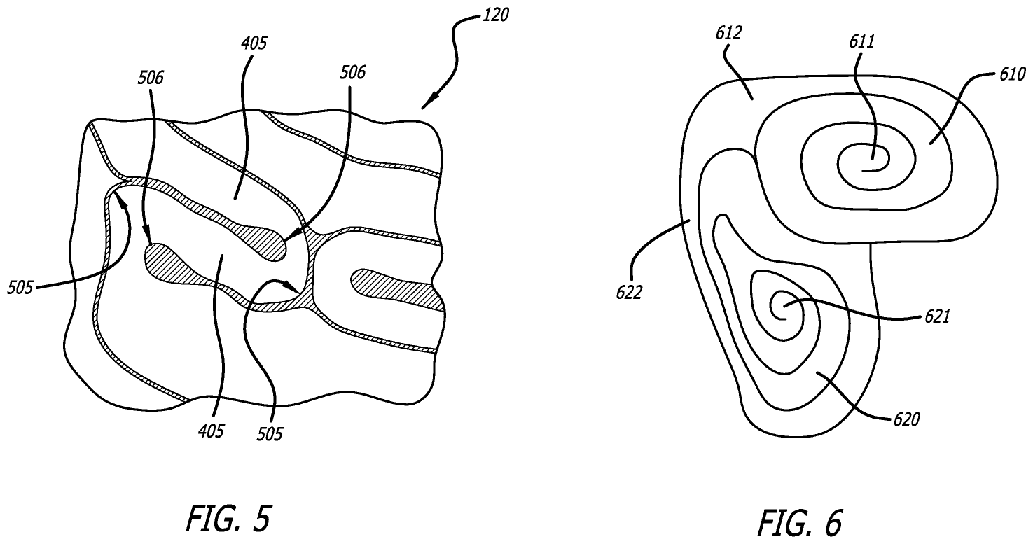

FIG. 6 illustrates an exemplary optional spiral arrangement of the flow channels that may be included in the pad 120. The illustrated embodiment shows a pair of interconnected spiraled flow channels 610, 620. The spiraled flow channel 610 extends between a central end 611 and a permitter end 612. The spiraled flow channel 620 extends between a central end 621 and a permitter end 622. The permitter end 622 is coupled with the permitter end 621 so that the spiraled flow channels 610, 620 define a continuous flow path extending between the central end 621 and the central end 611. The spiraled arrangement of the flow channels prevents inside corner pockets and sharp outside corners, which, in turn, inhibit low-flow conditions of the TTM fluid 112. The flow channels 405 of the pad 120 may include one or more spiraled arrangements.

According to some embodiments, the pad 120 may include none, all, or any subset of the features shown in and described in relation to FIGS. 4B-6. In some embodiments, the system 100 may include any or all of the fluid agitators 251A-251C. In other embodiments, all of the fluid agitators 251A-251C may be omitted from the system 100.

According to some embodiments, methods of exchanging thermal energy with the patient may include all or any subset of the following steps or process as performed by the system 100. One embodiment of the method, the targeted temperature management (TTM) system may circulate a TTM fluid within a thermal contact pad applied to the patient, where the TTM fluid has a temperature defined by a TTM module of the system in accordance with a TTM therapy. The system may also agitate or otherwise cause a disturbance the TTM fluid within flow channels of the pad to enhance a thermal energy exchange between the TTM fluid and the patient.

In some embodiments of the method, the TTM system includes a fluid agitator operatively coupled with the TTM fluid, where the fluid agitator causes the agitation of the TTM fluid within the pad.

In some embodiments of the method, a fluid flow disrupting mechanism disturbs the TTM flow within the flow cannels of the pad to inhibit low-flow conditions of the TTM fluid within the flow channels of the pad.

In some embodiments of the method, fluid flow disrupting members protrude within the flow channels, where the disrupting members are configured to enhance a TTM fluid flow velocity through otherwise low-flow areas of the flow channels.

In some embodiments, the method may include deflecting one or more disrupting members to cause the agitation or disturbance of the TTM fluid, where the deflection is caused by a force applied to the disrupting member by the TTM fluid. In some embodiments, a number of disrupting members may rotate to cause the agitation or disturbance of the TTM fluid, where the rotation results from a torque applied to the disrupting member by the TTM fluid flow.

In some embodiments of the method, a number of disrupting members may be formed of a material having an enhanced thermal conductivity, such as a thermal conductivity greater than a thermal conductivity of the channel wall material or the TTM fluid. As such, the thermal energy exchange may be at least partially defined by conductive heat transfer along the disrupting members.

In some embodiments of the method, a number of disrupting members may be formed of a material having an enhanced thermal compacity, such as a thermal compacity greater than a thermal compacity of the channel wall material and/or the TTM fluid. As such, a consistency and or stability of the thermal energy exchange may be at least partially defined by the thermal compacity of the disrupting members.

In some embodiments, the method may include drawing air into the flow channels via a number of orifices extending through an exterior wall of the pad between the TTM fluid and the environment, where the bubbles formed with the TTM fluid cause a flow disturbance of the TTM fluid to agitate the TTM fluid.

In some embodiments of the method, the TTM fluid may include a surfactant to enhance a thermal energy exchange between the TTM fluid and an inside surface of the flow channels.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A targeted temperature management (TTM) system for exchanging thermal energy with a patient, comprising:
a TTM module configured to provide a TTM fluid at a defined fluid temperature in accordance with a TTM therapy;
one or more thermal-contact pads fluidly coupled with the TTM module via a fluid delivery line (FDL) extending between the TTM module and the pad, the pad configured to:
receive the TTM fluid from the TTM module, and
circulate the TTM fluid within flow channels of the pad to define a thermal energy exchange between the TTM fluid and the patient, and
a fluid agitator operatively coupled with the TTM fluid, the fluid agitator configured to cause an agitation of the TTM fluid within the pad.

2. The system of claim 1, wherein the agitator causes an oscillation of the TTM fluid at a frequency greater than 20 KHz.

3. The system of claim 1, wherein the agitator is coupled with the TTM fluid at the pad.

4. The system of claim 1, wherein the agitator is coupled with the TTM fluid within a TTM fluid supply tank of the TTM module.

5. The system of claim 1, wherein the pad includes a fluid flow disrupting mechanism configured to inhibit low-flow conditions of the TTM fluid within the flow channels of the pad.

6. The system of claim 5, wherein the fluid flow disrupting mechanism includes a number of fluid flow disrupting members protruding within the flow channels, the disrupting members configured to enhance a TTM fluid flow velocity through otherwise low-flow areas of the flow channels.

7. The system of claim 6, wherein at least a first subset of the number of disrupting members are configured to deflect in response to a force applied by the TTM fluid flow.

8. The system of claim 6, wherein at least a second subset of the number of disrupting members are configured to rotate in response to a torque applied by the TTM fluid flow.

9. The system of claim 6, wherein at least a third subset of the number of disrupting members are formed of a material having a thermal conductivity greater than a thermal conductivity of the TTM fluid.

10. The system of claim 6, wherein at least a fourth subset of the number of disrupting members are formed of a material having a thermal compacity greater than a thermal compacity of the TTM fluid.

11. The system of claim 6, wherein:
the fluid flow disrupting mechanism includes a number of orifices extending through an exterior wall of the pad between the TTM fluid and the environment,
a negative pressure of the TTM fluid within the flow channels draws air through the orifices into the flow channels causing air bubbles within the flow channels, and
the air bubbles cause a flow disturbance of the TTM fluid to agitate the TTM fluid.

12. The system of claim 1, wherein the flow channels include a spiral flow path extending between a first end located at a central portion of the spiral and a second end located at a perimeter of the spiral.

13. The system of claim 1, wherein the TTM fluid includes a surfactant to enhance a thermal energy exchange between the TTM fluid and an inside surface of the flow channels.

14. A targeted temperature management (TTM) system for exchanging thermal energy with a patient, comprising:
a TTM module configured to provide a TTM fluid at a defined fluid temperature in accordance with a TTM therapy;
one or more thermal-contact pads fluidly coupled with the TTM module via a fluid delivery line (FDL) extending between the TTM module and the pad, the pad configured to:
receive the TTM fluid from the TTM module, and
circulate the TTM fluid within flow channels of the pad to define a thermal energy exchange between the TTM fluid and the patient,
wherein the pad includes a fluid flow disrupting mechanism configured to inhibit low-flow conditions of the TTM fluid within flow channels of the pad.

15. The system of claim 14, wherein the fluid flow disrupting mechanism includes a number of fluid flow disrupting members protruding within the flow channels, the disrupting members configured to enhance a TTM fluid flow velocity through otherwise low-flow areas of the flow channels.

16. The system of claim 15, wherein at least a first subset of the number of disrupting members are configured to deflect in response to a force applied by the TTM fluid flow.

17. The system of claim 15, wherein at least a second subset of the number of disrupting members are configured to rotate in response to a torque applied by the TTM fluid flow.

18. The system of claim 15, wherein at least a third subset of the number of disrupting members are formed of a material having a thermal conductivity greater than a thermal conductivity of the TTM fluid.

19. The system of claim 15, wherein at least a fourth subset of the number of disrupting members are formed of a material having a thermal compacity greater than a thermal compacity of the TTM fluid.

20. The system of claim 15, wherein:
the fluid flow disrupting mechanism includes a number of orifices extending through an exterior wall of the pad between the TTM fluid and the environment,
a negative pressure of the TTM fluid within the flow channels draws air through the orifices into the flow channels causing air bubbles within the flow channels, and
the air bubbles cause a flow disturbance of the TTM fluid to agitate the TTM fluid.

21. The system of claim 14, wherein the flow channels include a spiral flow path extending between a first end located at a central portion of the pad and a second end located at a perimeter of the pad.

22. The system of claim 14, wherein the TTM fluid includes a surfactant to enhance a thermal energy exchange between the TTM fluid and an inside surface of the flow channels.

23. The system of claim 14, further comprising a fluid agitator operatively coupled with the TTM fluid, the fluid agitator configured to cause an agitation of the TTM fluid within the pad.

24. The system of claim 23, wherein the agitator causes an oscillation of the TTM fluid at a frequency exceeding 20 KHz.

25. The system of claim 23, wherein the fluid agitator is coupled with the TTM fluid at the pad.

26-35. (canceled)
